(19) United States
(12) Patent Application Publication
(10) Pub. No.: US 2006/0002909 A1
(43) Pub. Date: Jan. 5, 2006

(54) Microorganism separated from Kefir grains, a microorganism culture obtained by culturing said microorganism or microorganisms including it, and a product using such microorganisms or microorganism cultures

(75) Inventor: Kazunori Takeda, Matsutou-shi (JP)

Correspondence Address: KODA & ANDROLIA, 2029 CENTURY PARK EAST, SUITE 1140, LOS ANGELES, CA, 90067, US

(73) Assignee: Kabushiki Kaisha Yonezawa Biru Shisutemu Sabisu

(21) Appl. No.: 11/151,847

(22) Filed: Jun. 13, 2005

(30) Foreign Application Priority Data
Jun. 14, 2004 (JP) 2004-175969

(51) Int. Cl. A61K 45/00; C12N 1/20; A23K 1/165

(52) U.S. Cl. 424/93.45; 435/252.9; 424/442

(57) ABSTRACT

A single or mixed culture performed under conditions in which a new species of the Lactobacillaceae family having antioxidant properties and separated from Kefir grains can be cultured. An antioxidant having the obtained culture or bacteria as active principles is formulated and used as a raw material for or added to drinks, foods, cosmetics or animal feeds.

### BACKGROUND OF THE INVENTION

1. Technical Field

The present invention relates to an antioxidant microorganism derived from a natural substance, used as a raw material of, or added to, drinks, foods, cosmetics and animal feeds, thereby preventing their deterioration due to oxidation, and scavenging or inhibiting generation of active oxygens and free radicals that are generated in vivo of humans and animals that use such drinks, foods, cosmetics or animal feeds. The present invention also relates to a microorganism culture obtained by culturing said microorganism or microorganisms including it. The present invention further relates to drinks, foods, cosmetics and animal feeds using such microorganisms or microorganism cultures.

2. Description of the Related Art

Lifestyle-related diseases such as malignant neoplasm (cancer), cerebrovascular diseases and diabetes are among the top causes of death in recent years. Major causes of lifestyle-related diseases include active oxygens and free radicals. There are different kinds of antioxidants: those added to drinks, foods, cosmetics and animal feeds to prevent their deterioration and preserve their quality; and those used to scavenge or inhibit generation of active oxygens and free radicals that are generated in vivo.

When oils and fats contained in a drink, food, cosmetic or animal feed product are oxidized, they not only affect the product's color and flavor, but they also cause the product's color to brown or fade and its nutrition value to decline. For this reason, many drinks, foods, cosmetics and animal feeds are added with antioxidants to preserve their quality.

Antioxidants used for such purposes in the fields of drinks, foods and animal feeds include natural antioxidants such as L-ascorbic acid (vitamin C), erythorbate (isoascorbic acid) and tocopherol (vitamin E) as well as phenolic synthetic antioxidants such as Di-n-butyl hydroxytoluene (BHT) and butylated hydroxyanisole (BHA).

L-ascorbic acid easily dissolves in water and is often used for its strong oxido-reduction properties. It is, however, difficult to be used in edible oil and fat because its acid (acidity) changes the taste of the food. For this reason, the use of L-ascorbic acid is limited to processed fruits and pickles, etc. Tocopherols are often used because of their ability to prevent unnecessary oxidation of oil ingredients. Their antioxidant properties, however, are not very strong, so they need to be used in combination with other antioxidants.

Natural antioxidants are generally inferior to synthetic antioxidants with respect to their antioxidant properties. On the other hand, while synthetic antioxidants such as Di-n-butyl hydroxytoluene (BHT) and butylated hydroxyanisole (BHA) have strong antioxidant properties, they are considered dubious food additives because of their cancer-inducing properties as exemplified by gene injury, mutagenicity and abnormal chromosomes. Their safety has been questioned.

Both natural and synthetic antioxidants are used in cosmetics to prevent oxidation of oils and fats used in them. But these antioxidants are considered to have adverse effects on the skin; they cause rough skin. Their safety has also been questioned.

For the above reasons, development of an antioxidant that has strong antioxidant properties and is highly versatile and derived from a natural substance has been desired.

Natural substances that have been reported to exhibit antioxidant properties include browning substances, amino acids, sugar alcohols, fructose (fruit sugar), fruit peels, wasabi (Japanese horseradish) and catechins. Antioxidants derived from microorganism cultures include lactic acid bacteria extracts that exhibit antioxidant effects.

In the past, these antioxidants were added to drinks, foods, cosmetics and animal feeds for the purposes of preventing their deterioration and preserving their quality. These antioxidants have also been found to scavenge or inhibit generation of active oxygens and free radicals that are generated in vivo. To take advantage of these properties, natural extracts with strong in vivo antioxidant properties have been sought.

Four kinds of active oxygens are known that are generated by in vivo oxidation. They are: singlet oxygens, super oxides, hydroxyl radicals and hydrogen peroxides. More than ten kinds of free radicals including alkyl hydro peroxides and hydro-peroxyl radicals are known that are generated by in vivo oxidation. The antioxidant properties can be detcrmined by measuring the radical-scavenging capacity using 1,1-diphenyl-2-picrylhydrazine (DPPH).

In vivo oxidation is considered to be a cause that triggers aging, lifestyle-related diseases and cancers. Aging causes weakening of physical functions not so much as sickness, such as hypokinesis, hypofunction of internal organs, failing memory, cataract and wrinkles. It also causes blood vessels to deteriorate, increasing the vulnerability to arterial sclerosis. Lifestyle-related diseases that are considered especially relevant to in vivo oxidation include arterial sclerosis and diabetes, as well as liver damage and rheumatic arthritis.

As for the relevance to cancer, in vivo oxidation is considered to be one of the causes that transmute and damage the substances that constitute DNA and mutate other cells when they are regenerated. In order to prevent such diseases, antioxidants that prevent or inhibit in vivo oxidation are drawing attention. They are researched and developed in the forms of foods with function claims, foods for specified health uses and supplements.

Antioxidants for such purposes include vegetable derivatives such as carotenoids (beta carotene, lycopene, fucoxanthine), phenolic compounds (flavonoid, anthocyanin, catechin), sulphides and beta diketones, and microorganism cultures such as DHA (docosahexaenoic acid) and EPA (eicosapentaenoic acid).

### BRIEF SUMMARY OF THE INVENTION

One object of the present invention is to provide a microorganism that is a natural extract with strong antioxidant properties, highly versatile for drinks, foods, cosmetics and animal feeds, and capable of maintaining the product's original taste and flavor while having in vivo antioxidant properties, as well as providing a. microorganism culture that is obtained by culturing this microorganism or microorganisms. Another objective of the present invention is to provide various drinks, foods, cosmetics and animal feeds that use such microorganisms.

Kefir is a traditional compound fermented milk product of lactic acid bacteria and yeast organisms thought to have originated in Caucasus, and is made by culturing Kefir grains in milk.

The inventor of the present invention discovered that natural Kefir grains make safe products for humans and animals when used in drinks, foods, cosmetics and animal feeds, and continued research into various uses of Kefir. The inventor further discovered that a cultured liquid made by culturing Kefir grains in a green-tea extract and removing bacteria from the microorganism culture had a skin-whitening effect and that Kefir grains themselves had antioxidant properties. Based on these findings, the inventor applied for a patent in the form of a cosmetic that takes advantage of such properties of Kefir. (Japanese Patent Application No. 2002-315520, Application filing date: Oct. 30, 2002).

As a result of further research, the inventor has identified a microorganism derived from a natural substance and having antioxidant properties that can be used as a raw material of, or added to, drinks, foods, cosmetics and animal feeds, thereby preventing their deterioration due to oxidation and scavenging or inhibiting generation of active oxygens and free radicals that are generated in vivo of humans and animals. The inventor has also discovered that a microorganism culture obtained by culturing said microorganism or microorganisms is capable of achieving the above objectives. The inventor has also come to develop a product having antioxidant properties by adding the microorganisms in drinks, foods, cosmetics and animal feeds.

The present invention relates to:

(1) a new species of microorganism having antioxidant properties that is separated from Kefir grains and belongs to Lactobacillaceae;

(2) a microorganism culture having antioxidant properties obtained in a single culture of a new species of microorganism that is separated from Kefir grains and belongs to Lactobacillaceae;

(3) a microorganism culture having antioxidant properties that is obtained by culturing microorganisms separated from Kefir grains;

(4) a drink having antioxidant effects added with cultures or bacteria obtained by culturing microorganisms separated from Kefir grains and/or a new species of microorganism belonging to Lactobacillaceae;

(5) a food having antioxidant effects added with cultures or bacteria obtained by culturing microorganisms separated from Kefir grains and/or a new species of microorganism belonging to Lactobacillaceae;

(6) a cosmetic having antioxidant effects added with cultures or bacteria obtained by culturing microorganisms separated from Kefir grains and/or a new species of microorganism belonging to Lactobacillaceae; and

(7) an animal feed having antioxidant effects added with cultures or bacteria obtained by culturing microorganisms separated from Kefir grains and/or a new species of microorganism belonging to Lactobacillaceae.

The “antioxidant properties” is defined as including the ability to prevent oxidization thereby preserving the quality of the product as well as the ability to scavenge or inhibit active oxygens and free radicals that are generated in vivo thereby preventing in vivo oxidization when given to humans or animals as drinks, foods or animal feeds. The “microorganism culture” means a culture of microorganisms or a single microorganism separated from Kefir grains.

The present invention has the effect of providing a new species of microorganism having antioxidant properties that is separated from Kefir grains and belongs to Lactobacillaceae. By using this microorganism, which has antioxidant properties, as a raw material for drinks, foods, cosmetics or animal feeds, or by adding it to these products or their raw materials, it is possible to prevent their deterioration due to oxidation and to scavenge or inhibit generation of active oxygens and free radicals that are generated in vivo of humans and animals that use them.

The present invention has the effect of providing a microorganism culture having antioxidant properties that includes a new species of microorganism separated from Kefir grains and belonging to Lactobacillaceae. This culture, which is not toxic and has no adverse effect, has a variety of applications including raw materials and additives for drinks, foods; cosmetics, animal feeds, etc.

The present invention has the effect of providing a microorganism culture having antioxidant properties that is obtained by culturing microorganisms separated from Kefir grains. This culture, which is not toxic and has no adverse effect, has a variety of applications including raw materials and additives for drinks, foods, cosmetics and animal feeds.

The present inventions has the effects of providing antioxidant drinks, foods, cosmetics and animal feeds that are added with cultures or bacteria obtained by culturing microorganisms separated from Kefir grains and/or a new species of microorganism belonging to Lactobacillaceae.

### BRIEF DESCRIPTION OF THE DRAWINGS

FIG. 1 is a graph showing the relationship between the culture time and the growth of the microorganism;

FIG. 2 is a graph showing the relationship between the culture time (growth of the microorganism) and the changes of Ph;

FIG. 3 is a graph showing the relationship between the culture time (growth of the microorganism) and the antioxidant properties;

FIG. 4 is a photograph of Gram stained SIID1719-6b; and

FIG. 5 shows the results of the molecular pedigree analysis of SIID1719-6b.

### DETAILED DESCRIPTION OF THE INVENTION

Preferred embodiments of the present invention will now be explained.

Strains of microorganisms separated from Kefir grains include Acetobacter cerevisiae SIID1719-2b, Gluconobacter oxydans SIID1719-3b, Lactobacillus sp. SIID1719-6b, which is a new species of the Lactobacillaceae family, Pichiamembrani faciens SIID1719-1y, Saccharomyces cerevisiae SIID1719-4y and Picchia anomala SIID1719-5y.

In order to find out whether microorganisms separated from Kefir grains and a microorganism culture obtained in a single culture of Lactobaccilus sp. SIID1719-6b, which is a new species of the strain Lactobacillaceae family, have antioxidant properties, we conducted the following evaluation tests. As a result, we found that these microorganisms and the Lactobacillus sp. SIID1719-6b itself have antioxidant properties. Moreover, active principles of the culture that is removed of bacteria in a centrifugal separator and sterilized using a sterilizing filter were also found to have antioxidant properties. According to the present invention, anything that shows the same properties in symbiosis with microorganisms whose safety with Lactobacillus sp. SIID1719-6b is confirmed can be used. The Lactobacillus sp. SIID1719-6b, which is a new species of the Lactobacillaceae family, is deposited at the International Patent Organism Depository of the National Institute of Advanced Industrial Science and Technology.

As a typical example, a strain of Lactobacillus sp. SIID1719-6b, which is a new species of the family Lactobacillaceae, was cultured and its antioxidant properties were evaluated as described in the following paragraphs.

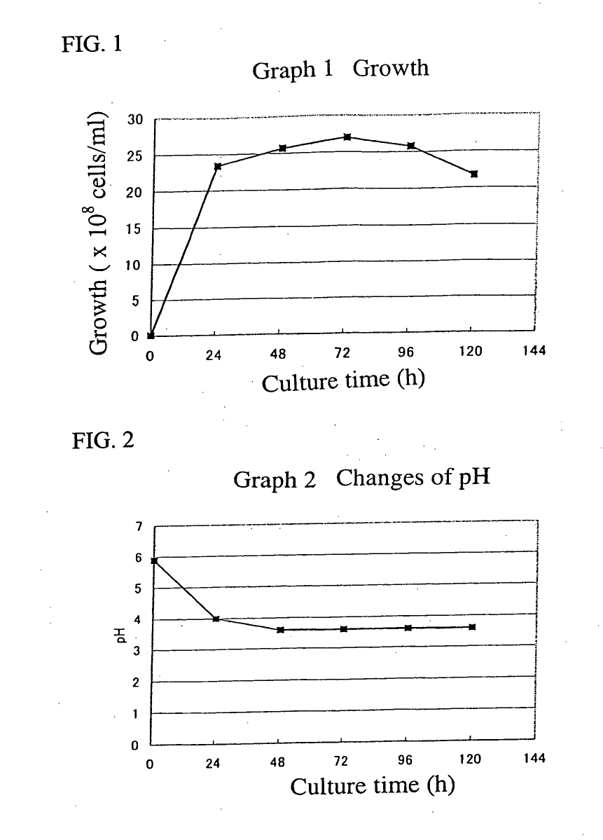

A strain of Lactobacillus sp. SIID1719-6b, which is a new species of the family Lactobacillaceae, is cultured in a medium M.R.S. Broth (Oxoid England, UK). The composition of the medium M.R.S. is shown in table 1.2×10⁶ cells/ml of bacteria are cultured statically at a culture temperature of 30° C. and sampled every 24 hours to measure the number of bacteria and pH. The growth changes in the number of bacteria are shown in FIG. 1, and the changes of pH are shown in FIG. 2. There is no limitation as to the medium composition, substrate and temperature of the culture as long as the conditions allow the bacteria to grow well.

TABLE 1
Composition of the MRS medium in 1 liter of purified water

| Ingredients of the medium | Amount added | |
|---|---|---|
| Peptone | 10.0 | g |
| Lab-Lemco' powder | 8.0 | g |
| Yeast extract | 4.0 | g |
| Glucose | 20.0 | g |
| Sorbitan mono-oleate | 1 | ml |
| Dipotassium hydrogen phosphate | 2.0 | g |
| Sodium acetate 3H₂O | 5.0 | g |
| Triammonium citrate | 2.0 | g |
| Magnesium sulphate 7H₂O | 0.2 | g |
| Manganese sulphate 4H₂O | 0.05 | g |

The inventors of the present application tested the antioxidant properties of the above culture. First, 190 μl of 0.1 mM DPPH (dissolved in 100% ethanol) is added to each of 96 wells on a microplate. Next, samples of the culture (0, 24, 48, 72, 96 and 120 hours) and 10 μl of the MRS medium (blank) are added to each well to start radical-scavenging reactions. The light absorption immediately after (0 minute) and 30 minutes after the addition of the samples were measured with a microplate reader. The radical-scavenging rate was calculated from the light absorption of the blanks and samples.

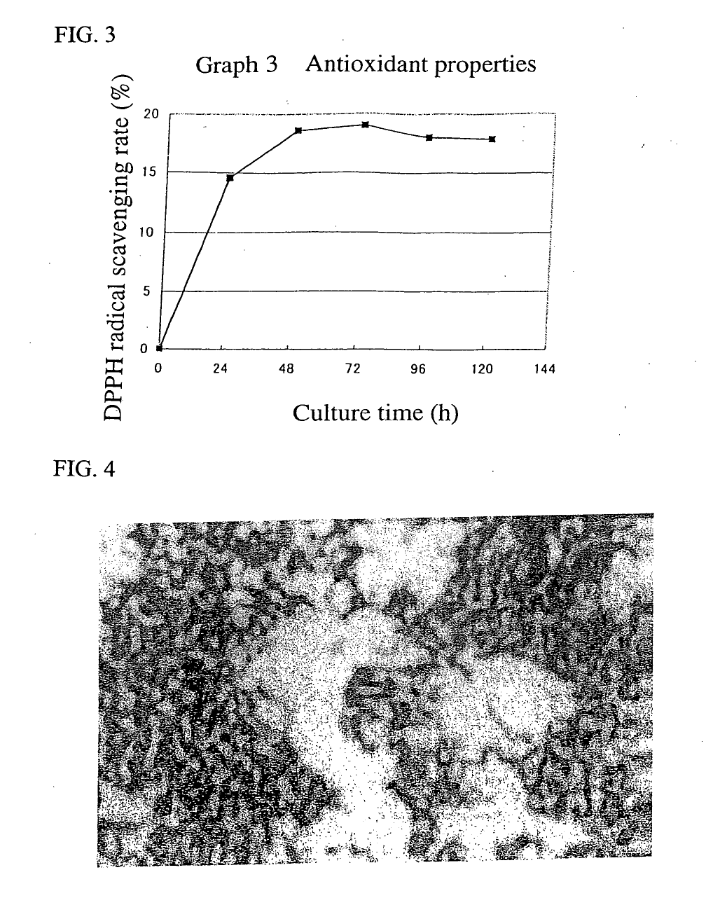

The results are shown in FIG. 3. As the culture time becomes longer, the microorganisms grow and their pH changes. As the microorganisms grow, the antioxidant properties also become more evident.

The inventors have identified Lactobaccilus sp. SIID1719-6b to be of a new species. Our method of identification and the results of the microorganisms test are described in the following paragraphs.

As a first-stage bacteria test, we observed the cell shape, Gram stainability, the existence of spores and the motility of flagella using an optical microscope (U-LH1000, Olympus, Japan). We also observed the shapes of colonies on an MRS Broth (Oxoid, England, UK)+agar. We tested for catalase reactions, oxidase reactions, acid and gas generation from glucose and oxidation and fermentation of glucose.

The results of the mycological properties test are shown in table 2, and a photo of a Gram stained culture is shown in FIG. 4.

TABLE 2
Results of the mycological properties test

| Test item | | | Result |
|---|---|---|---|
| | Cell shape | Bacillus | |
| | | (06-0.7 × 1.5-2.0 μm) | |
| | Gram stain | | + |
| | Spore | | − |
| | Motility | | − |
| | | Culture time: 24 h | |
| | | Circular | |
| | Colony shape | Smooth edges | |
| | | Convex | |
| | | Glossy | |
| | | Milk white | |
| | Culture temperature ° C. | 37 | + |
| | | 45 | − |

| | |
|---|---|
| Catalase | − |
| Oxidase | − |
| Acid/gas generation (glucose) | +/− |
| Oxidation-fermentation (O/F) test | +/+ |
| (glucose) | |

The inventors conducted a physiological and biochemical test. The results are shown in table 3.

Physiological and biological test results

| Ingredients | Result | Ingredients | Result | Ingredients | Result |
|---|---|---|---|---|---|
| Glycerol* | − | Mannitol* | + | Raffinose* | − |
| Erythritol* | − | Sorbitol* | + | Starch* | − |
| D-arabinose* | − | α-methyl-D-mannose* | − | Glycogen* | − |
| L-arabinose* | − | α-methyl-D-glucose* | + | Xylitol* | − |
| Ribose* | − | N-acetylglucosamine* | + | Gentiobiose* | + |
| D-xylose* | − | Amygdalin* | − | D-Turanose* | + |
| L-xylose* | − | Arbutin* | + | D-Lyxose* | − |
| Adonitol* | − | Esculin* | + | D-tagatose* | − |
| β-methyl-D-xylose* | − | Salicin* | + | D-fucose* | − |
| Galactose* | − | Cellobiose* | − | L-fucose* | − |
| Glucose* | + | Maltose* | − | D-arabitol* | − |
| Fructose* | + | Lactose* | − | L-arabitol* | − |
| Mannose* | + | Melibiose* | − | Gluconate* | − |
| Sorbose* | + | Saccharose* | + | 2-ketogluconic | − |
| | | | | acid* | |
| Rhamnose* | + | Trehalose* | + | 5-ketogluconic | − |
| | | | | acid* | |
| Dulcitol* | − | Inulin* | − | | |
| Inositol* | − | Melezitose* | − | | |

The inventors used the PrepMan Method (Applied Biosystems, CA, USA) for extraction of genome DNAs. Using the extracted genome DNAs as templates, we amplified the region of approximately 1500 to 1600 bp of all of the base sequence of 16S Ribosomal RNA genes (16S rDNA) by PCR. After that, we sequenced the amplified 16S rDNAs and obtained a base sequence. For purification of PCR products and cycle sequence, we used the MicroSeq Full 16S rDNA Bacterial Sequencing Kit (Applied Biosystems, CA, USA) (MicroSeq is a registered trademark). For the thermal cycler, we used the GeneAmp PCR SYSTEM 9600(Applied Biosystems, CA, USA), and for the DNA sequencer, we used the ABI PRISM 3100 DNA Sequencer (Applied Biosystems, CA, USA). For the basic operations from the extraction of genome DNAs to cycle sequence, we followed the AppliedBiosystems' protocol (P/N4308132 Rev. A).

The base sequence of 16S rDNA of SIID1719-6b is shown in table 4.

TABLE 4

(16S rDNA base sequence of SIID1719-6b)
Sequence length: 1521
Sequence type: DNA
Name of organism: Lactobacillus mali

Sequence

| | |
|---|---|
| gagtttgatc ctggctcagg acgaacgctg gcggcgtgcc | 50 |
| taatacatgc | |
| aagtcgtacg cagtttcttc accgagtgct tgcactcacc | 100 |
| gaagaaactg | |
| agtggcgaac gggtgagtaa cacgtgggta acctgcccaa | 150 |
| aagaggggga | |
| taacacttgg aaacaggtgc taataccgca taacaacaaa | 200 |
| aaccgcctgg | |
| tttttgttta aaagatggtt tcggctatca cttttggatg | 250 |
| gacccgcggc | |
| gtattagcta gttggtaagg taatggctta ccaaggcagt | 300 |
| gatacgtagc | |
| cgaactgaga ggttgatcgg ccacattggg actgagagac | 350 |
| ggcccaaact | |
| cctacgggag gcagcagtag ggaatcttcc acaatggacg | 400 |
| caagtctgat | |
| ggagcaacgc cgcgtgagtg aagaaggttt tcggatcgta | 450 |
| aaactctgtt | |
| gttagagaag aacgtgtgtg agagtaactg ttcatrcagt | 500 |
| gacggtatct | |
| aaccagaaag ccacggctaa ctacgtgcca gcagccgcgg | 550 |
| taatacgtag | |
| gtggcaagcg ttgtccggat ttattgggcg taaagggaac | 600 |
| gcaggcggtc | |
| ttttaagtct gatgtgaaag ccttcggctt aaccgaagtc | 650 |
| gtgcattgga | |
| aactgggaga cttgagtgca gaagaggaga gtggaactcc | 700 |
| atgtgtagcg | |
| gtgaaatgcg tagatatatg gaagaacacc agtggcgaaa | 750 |
| gcggctctct | |
| ggtctgtaac tgacgctgag gttcgaaagc gtgggtagca | 800 |
| aacaggatta | |
| gataccctgg tagtccacgc tgtaaacgat gaatgctaag | 850 |
| tgttggaggg | |
| tttccgccct tcagtgctgc agctaacgca ttaagcattc | 900 |
| cgcctgggga | |
| gtacgaccgc aaggttgaaa ctcaaaggaa ttgacggggg | 950 |
| cccgcacaag | |
| cggtggagca tgtggtttaa ttcgaagcaa cgcgaagaac | 1000 |
| cttaccaggt | |
| cttgacatct tttgctaacc tgagagatca ggygttccct | 1050 |
| tcggggacaa | |
| aatgacaggt ggtgcatggt tgtcgtcagc tcgtgtcgtg | 1100 |
| agatgttggg | |
| ttaagtcccg caacgagcgc aacccttatt actagttgcc | 1150 |
| agcatttagt | |
| tgggcactct agtgagactg ccggtgacaa accggaggaa | 1200 |
| ggtggggatg | |
| acgtcaaatc atcatgcccc ttatgacctg ggctacacac | 1250 |
| gtgctacaat | |
| ggacggtaca acgagtcgca agacogogag gtttagctaa | 1300 |
| tctcttaaaa | |
| ccgttctcag ttcggattgt aggctgcaac tcgcctacat | 1350 |
| gaagtcggaa | |
| tcgctagtaa tcgcggatca gcatgccgcg gtgaatacgt | 1400 |
| tcccgggcct | |
| tgtacacacc gcccgtcaca ccatgagagt ttgtaacacc | 1450 |
| caaagccggt | |
| gcggtaacct tttaggagcc agccgtctaa ggtgggacag | 1500 |
| atgattgggg | |
| tgaagtcgta acaaggtagc c | 1521 |

Using BLAST, we conducted a homology search of the above base sequence by comparing it against the DNA base sequence database, GenBank. From the base sequence of the 16S rDNA that has been obtained, we conducted a homology search for species that are considered to be close relatives of the samples and identified the top five strains. The results of the homology search are shown in table 5.

TABLE 5

| | | As of | |
|---|---|---|---|
| Homology search | | May 10, 2004 | |
| results Entries | Strains | Accession No. | Identity |
| Lactobacillus mali | | M58824 | 1424/1495 = 95.3% |
| Lactobacillus sp. | CECT5924 | AJ576009 | 1364/1407 = 96.9% |
| CECT5924 | | | |
| Lactobacillus nageli | NRIC0559 | AB162131 | 1440/1511 = 95.3% |
| Lactobacillus sp. | | AB016864 | 1317/1364 = 96.6% |
| Pediococcus | DSM20331 | AJ318414 | 1351/1423 = 94.9% |
| damnosus | | | |

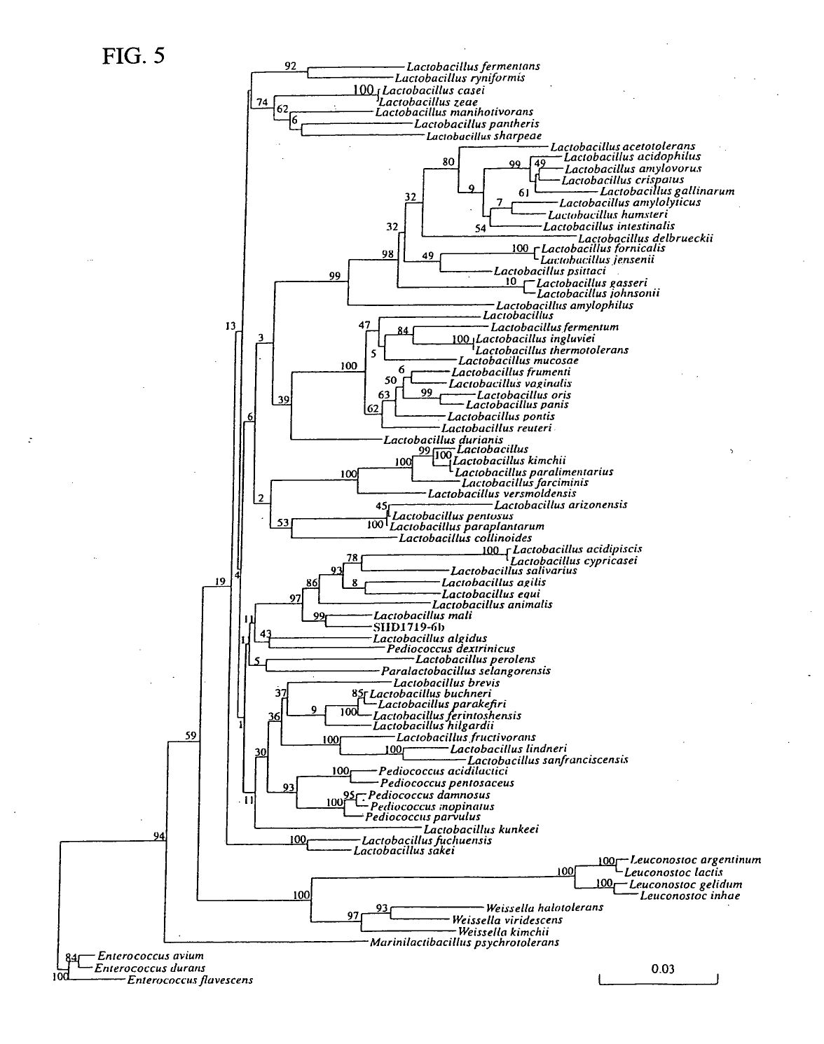

Based on the information obtained so far, we constructed a molecular pedigree using the 16S rDNA base sequence and the 16S rDNA base sequence of strains that are considered to be their close relatives. We downloaded the base sequence alignment and the 16S rDNAs used in the construction of the molecular pedigree from the GenBank/DDBJ/EMBL on the basis of the strain-derivative alignment. For computation of the molecular pedigree, we used the neighbor-joining method, in which 1,000 bootstraps were generated to test the validity of the topology. For analysis, we used DNASIS Pro (Hitachi Software Engineering, Yokohama). The analysis results of the molecular pedigree are shown in FIG. 5.

The inventors extracted and purified DNAs from cultured bacteria, and obtained DNAs for measuring the G+C content. We used High Performance Liquid Chromatography (HPLC). We added 20 liters of nuclease P1 solution (0.1 mg/mL of nuclease P1 (DNA-GC Kit, made by Yamasa Corporation, sold by Seikagaku Corporation), 40 mM of sodium acetate and 2 mM of zinc sulfate [pH 5.3]) to the same amount of DNAs (350 g/ml) that are dissolved in sterilized water and thermally denatured, and treated the solution for one hour at 50° C. to dissolve it into nucleotides and used it as a sample for HPLC measurement. We performed measurement using an HPLC device (LC-10, Shimadzu, Kyoto) under the following conditions.

Moving phase: 10 mM phosphate buffer (10 mM potassium phosphate, 10 mM monobasic potassium phosphate [pH 7.0]).

Column: Develosil RPAQUEOUS, φ4.6 mm×250 mm (Nomura Chemical, Co., Ltd., Aichi).

Flow speed: 1.5 m/min.

Detected wavelength: 270 mm.

The inventors identified each nucleotide from the retention times of the standard nucleotide compound (containing equal moles of dCMP, dAMP, dGMP and dTMP) contained in the DNA-GC Kit (made by Yamasa Corporation, sold by Seikagaku Corporation) and the sample. At the same time, we calculated the DNA base composition of the sample expressed as a GC content from the ratio of the peak areas of the standard nucleotide compound and the sample using the following equation.
G+C mol %=((Cx/Cs+Gx/Gs)/(Cx/Cs+Ax/As+Gx/Gs+Tx/Ts))×100

Nx: Value of the peak area of dCMP, dAMP, dGMP and dTMP of the sample.

Ns: Value of the peak area of dCMP, dAMP, dGMP and dTMP contained in the standard nucleotide compound.

The results of the G+C content measurement are shown in Table 6.

TABLE 6
G + C content measurement results

| Sample | GC content (%) |
|---|---|
| 1719-6b | 40.5 |

According to the analysis of the 16S rDNA base sequence, the SIID1719-6b's similarity to its closest relative strain Lactobacillus mali is 95.3%. While the G+C content of Lactobacillus mali is 32.5-33.0%, that of SIID1719-6b is 40.5%. In taxonomy, there is a guideline for two species to be considered the same species. According to this guideline, in order for any two species to be considered the same species, the result of their DNA-DNA hybrid formation test must be 70% or higher. That is the definition of “being the same species”. In order for the result of a DNA-DNA hybrid formation test to be 70% or higher, the result of a 16S rDNA base sequence analysis must exceed 97% and the difference in the G +C content between the two species compared must be smaller than 3%. Our test results do not meet these criteria. Accordingly, it has been found that SIID1719-6b is a new species.

1. A new species of microorganism having antioxidant properties that is separated from Kefir grains and belongs to Lactobacillaceae.

2. A microorganism culture having antioxidant properties obtained in a single culture of a new species of microorganism that is separated from Kefir grains and belongs to Lactobacillaceae.

3. A microorganism culture having antioxidant properties that is obtained by culturing microorganisms separated from Kefir grains.

4. A drink having antioxidant effects added with cultures or bacteria obtained by culturing microorganisms separated from Kefir grains and/or a new species of microorganism belonging to Lactobacillaceae.

5. A food having antioxidant effects added with cultures or bacteria obtained by culturing microorganisms separated from Kefir grains and/or a new species of microorganism belonging to Lactobacillaceae.

6. A cosmetic having antioxidant effects added with cultures or bacteria obtained by culturing microorganisms separated from Kefir grains and/or a new species of microorganism belonging to Lactobacillaceae.

7. An animal feed having antioxidant effects added with cultures or bacteria obtained by culturing microorganisms separated from Kefir grains and/or a new species of microorganism belonging to Lactobacillaceae.


SEQUENCE LISTING (1 sequence)

SEQ ID NO: 1 - DNA, 1521 bp, Lactobacillus mali
gagtttgatcctggctcaggacgaacgctggcggcgtgcctaatacatgcaagtcgtacgcagtttcttcaccgagtgcttgcactcaccgaagaaactgagtggcgaacgggtgagtaacacgtgggtaacctgcccaaaagagggggataacacttggaaacaggtgctaataccgcataacaacaaaaaccgcctggtttttgtttaaaagatggtttcggctatcacttttggatggacccgcggcgtattagctagttggtaaggtaatggcttaccaaggcagtgatacgtagccgaactgagaggttgatcggccacattgggactgagacacggcccaaactcctacgggaggcagcagtagggaatcttccacaatggacgcaagtctgatggagcaacgccgcgtgagtgaagaaggttttcggatcgtaaaactctgttgttagagaagaacgtgtgtgagagtaactgttcatrcagtgacggtatctaaccagaaagccacggctaactacgtgccagcagccgcggtaatacgtaggtggcaagcgttgtccggatttattgggcgtaaagggaacgcaggcggtcttttaagtctgatgtgaaagccttcggcttaaccgaagtcgtgcattggaaactgggagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgctgtaaacgatgaatgctaagtgttggagggtttccgcccttcagtgctgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgctaacctgagagatcaggygttcccttcggggacaaaatgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggatgacgtcaaatcatcatgccccttatgacctgggctacacacgtgctacaatggacggtacaacgagtcgcaagaccgcgaggtttagctaatctcttaaaaccgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtgcggtaaccttttaggagccagccgtctaaggtgggacagatgattggggtgaagtcgtaacaaggtagcc